(19) United States
(12) Patent Application Publication
(10) Pub. No.: US 2009/0054963 A1
(43) Pub. Date: Feb. 26, 2009

(54) CARDIAC PACEMAKER - ELECTRODE WITH HELICAL SCREW

(76) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 12/192,373

(22) Filed: Aug. 15, 2008

(30) Foreign Application Priority Data
Aug. 22, 2007 (DE) 102007039553.3-54

(51) Int. Cl. A61N 1/05

(52) U.S. Cl. 607/127

(57) ABSTRACT

A cardiac pacemaker—electrode (1) provided at its distal end with a helical screw (2), connected to an electrically conductive supply helix (5), which in turn can be coupled to a cardiac pacemaker via a connector (5) and a connector pin (6) and in turn can be rotated for rotating and axially adjusting the helical screw (2). A stylet (8) can be inserted through an interior longitudinal cavity of the supply helix (4), and includes a distal end (9) that can be directly or indirectly connected or coupled to the rotational helical screw (2), with the stylet (8) additionally being directly or indirectly coupled in a rotationally fixed fashion to the proximal end of the supply helix (4) so that when the stylet (8) is rotated both the supply helix (4) as well as the helical screw (2) are rotated without any considerably different rotation of the supply helix (4) occurring.

### CROSS-REFERENCE TO RELATED APPLICATIONS

This application claims the benefit of German Application DE 10 2007 039 553.3, filed Aug. 22, 2007, which is incorporated herein by reference as if fully set forth.

### BACKGROUND

The invention relates to a cardiac pacemaker—electrode with a helical screw, displaceable by rotation from a retracted position into an active holding position and in its holding position engaging the heart tissue, and which is connected to an electrically conducting feed helix, which in turn can be coupled to a cardiac pacemaker via a connector and which in turn is rotational for rotating and axially adjusting the helical screw.

Such a cardiac pacemaker—electrode is known in various forms and arrangements and has, due to the helical screw serving to screw into the heart tissue, provided a good contact and results in a good fastening which can withstand the constant cardiac motion. One example for such a cardiac pacemaker—electrode is described in U.S. Pat. No. 4,624,266.

It is disadvantageous that during the rotation of the helical screw, the surgeon, based on the torsion of the supply helix, cannot determine with sufficient certainty if the relatively numerous rotations of this supply helix at the side of its connector has resulted in a sufficiently long projection and rotation of the helical screw with an appropriately expansive engagement depth in the heart. In order to determine if the helical screw has been axially displaced sufficiently far from its initial position a control must be performed via X-Rays.

### SUMMARY

Therefore, the object is to provide a cardiac pacemaker—electrode of the type mentioned at the outset by which a sufficient axial adjustment of the helical electrode can be performed in a controlled fashion without requiring any X-Ray procedure.

In order to attain this object the cardiac pacemaker—electrode defined at the outset is characterized in that the supply helix has a clear interior cross-section, that in the operational state a rotational stylet can be inserted into the supply helix, which with its distal end can be coupled in a form-fitting manner to a rotational holder of the helical screw, and that the stylet can additionally be coupled in a rotationally fixed manner to the proximal end of the supply helix or to a part engaging the supply helix or to a rotational connector pin.

In this way, it is possible that the surgeon can perform the rotational motion necessary to axially adjust the helical electrode at the supply helix via the stylet both at its proximal end as well as its distal end, so that a torsion of the supply helix occurs to a very slight extent only or not at all and thus from the number of rotations performed at the proximal end it can be concluded how many threads of the helical screw have been projected from their initial position in the axial direction, which represents a measure for the engagement depth of the helical screw.

One preferred embodiment of the invention provides that the proximal end of the stylet is provided with a sheath-like coupling, which matches the connector pin connected to the supply helix in a rotationally fixed manner and can be connected thereto particularly in a removable or detachable manner. The rotational connector pin for rotating the supply helix and thus the helical screw can therefore be provided with an additional function, by serving as the coupling counterpart for a coupling arranged at the proximal end of the stylet so that the rotation of said coupling simultaneously rotates the connector pin and thus the supply helix, but also the stylet, which engages the holder of the helical spring immediately at the distal end. In this way, the rotational motion at the proximal end of the cardiac pacemaker—electrode is simultaneously transferred immediately to the helical electrode, which practically excludes any torsion of the supply helix rotated here.

The sheath-like coupling for the connector pin may be provided with a clamping sheath that can be coupled thereto in a detachable fashion, which in the operating position has a coupling appendage aligned to the proximal end, via which the proximal end of the stylet can be connected in the direction of rotation in a power transmitting fashion. Thus, in the operating state, this clamping sheath serving as a coupling can be connected to a rotational connector pin and coupled to the proximal end of the stylet so that a rotation of this coupling and/or the proximal end of the stylet simultaneously rotates the stylet and the connector pin and thus also the supply helix. The rotation of the stylet is here additionally transferred by said stylet immediately to the holder of the helical screw.

A coupling sheath can be provided connected to the proximal end of the stylet which concentrically encompasses the end of the stylet at a distance and can be attached to the coupling appendage of the sheath-like coupling in a force-fitting and/or form-fitting fashion. The stylet can therefore be inserted in the axial direction through the hollow connector pin and the supply helix and with its distal end connected to the holder of the helical screw in a form-fitting manner, by which simultaneously the coupling sheath located at the proximal end of the stylet is connected to the sheath-like coupling in a form-fitting or force-fitting manner, which in turn impinges the connector pin. Therefore, the user only needs to bring the stylet into the operating position in order to then allow by a rotation of the stylet simultaneously to rotate the connector pin and the supply helix as well as the holder for the helical screw, by which any torsion of the supply helix by said rotational motion can be avoided.

The distal end of the stylet can be embodied such that it can be coupled to the holder for the helical screw in a form-fitting manner in the direction of rotation and flattened or polygonal, for example as a square, hexagon, or similarly, and a matching coupling opening may be provided at the holder for the helical screw. Such an embodiment in the area of the distal end of a stylet is known per se and is used to allow for the rotation of the stylet to be immediately transferred to the holder for the helical screw. Due to the fact that this holder is also connected to the supply helix their rotation is to be performed simultaneously for the helical screw to remain in the projected position even in the position of operation.

It may be beneficial for the coupling appendage of the clamping sheath, engaging the connector pin in the operating position in a detachable fashion, and the interior cross-section of the coupling sheath arranged at the proximal end of the stylet to be profiled matching each other so that the coupling sheath and the coupling appendage can be displaced in the axial direction in a telescopic fashion and to be form-fitting in the rotational direction. The connection of the stylet to the coupling engaging the connector pin in a sheath-like fashion can therefore be a very simple plug-connection to be performed in the axial direction.

The coupling sheath arranged at the proximal end of the stylet can simultaneously serve or be embodied as a rotational handle and particularly be roughened or profiled at the outside. This way, this counter-coupling or coupling sheath arranged at the stylet has an additional function by simultaneously serving to compensate and transfer the rotary motion of a user.

A cardiac pacemaker—electrode results primarily in combinations of individual or several of the above-described features and measures in which the rotation of the supply helix for the axial adjustment of the helical screw during its engagement for example in heart tissue is supported by a stylet immediately engaging this holder, which engages both the holder as well as the proximal end of the supply helix, so that this supply helix is practically not subject to any torsion during the implanting and the axial adjustment of the helical screw by the rotational motion. With appropriate precision, the number of rotations performed allows conclusions to be made on the engagement depth of the helical screw so that a control of said engagement of the helical screw by X-Ray examination can be avoided.

### BRIEF DESCRIPTION OF THE SEVERAL VIEWS OF THE DRAWINGS

In the following, an exemplary embodiment of the invention is explained in greater detail using the drawing. Shown in a partially schematic illustration:

FIG. 1 is a side view of the proximal end of a cardiac pacemaker—electrode with a stylet being inserted coaxially in an interior longitudinal cavity of position, with simultaneously, a sheath-like coupling is additionally shown prior to the attachment on the connector pin and prior to the fastening there,

FIG. 2 is a longitudinal cross-sectional view of the distal end of the cardiac pacemaker—electrode according to FIG. 1, in a scale slightly enlarged in reference to FIG. 1, with the stylet already being inserted but not yet reaching its coupling or operating position at the holder of the helical electrode,

FIG. 3 a view according to FIG. 1, with the sheath-like coupling being mounted on the connector pin and engaging a counter coupling located at the stylet and engaging it in a form-fitting manner, with the stylet being displaced in the axial direction into its operating position, and

FIG. 4 is a longitudinal cross-sectional view according to FIG. 2, in an enlarged scale in reference to FIG. 3, with the distal end of the stylet being coupled to a matching opening of the holder for the helical electrode.

### DETAILED DESCRIPTION OF THE PREFERRED EMBODIMENTS

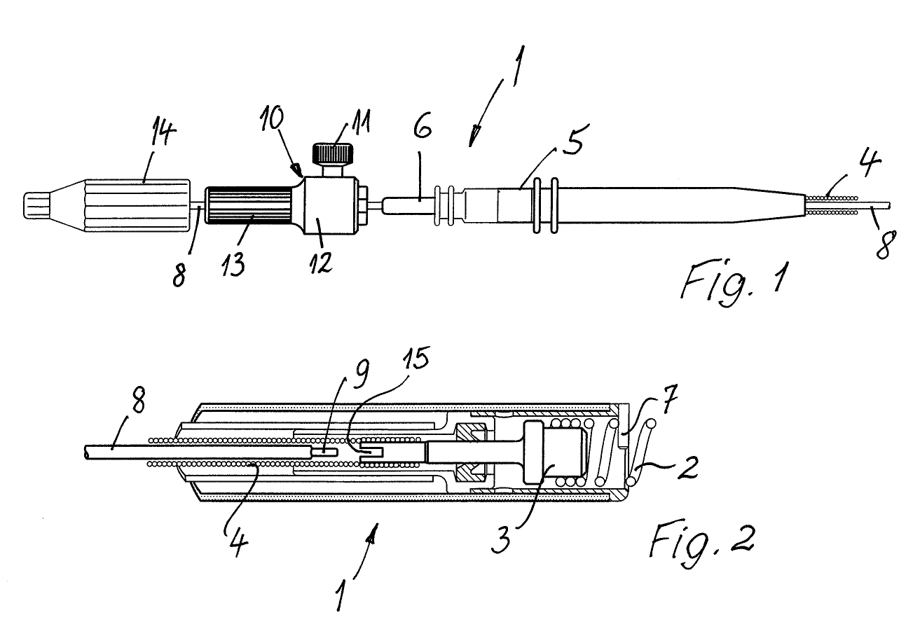

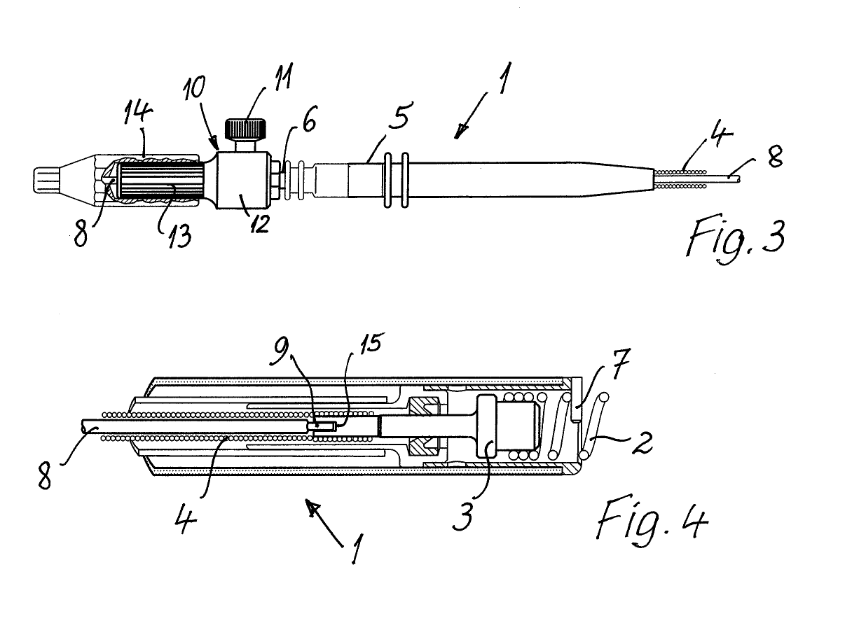

FIGS. 3 and 4 practically show a cardiac pacemaker—electrode, in its entirety marked 1, which here is however shown interrupted when observed together. While the operating state is shown in FIGS. 3 and 4, FIGS. 1 and 2 show the arrangement in a position and situation in which the matching parts remain to be pushed together axially in order to reach the operating position according to FIGS. 3 and 4.

Here, it is discernible that this cardiac pacemaker—electrode 1 has a helical screw 2, which is displaceable by rotation from a retracted position into an active fastening position, not yet reached in the figures, which can engage the tissue of the heart. The helical screw is held by a holder 3, via which it is connected to an electrically conductive supply helix 4.

This supply helix 4, in turn, can be coupled via a connector 5 and a corresponding connector 6, in a manner known per se, to a cardiac pacemaker, not shown in greater detail. In order to rotate and axially adjust the helical screw 2 and/or its holder 3 in reference to the engaging part 7, which engages windings of the helical screw 2, the supply helix 4 can be appropriately rotated in a manner to be described.

Primarily based on FIGS. 1 and 2, but also of FIGS. 3 and 4, i.e. a longitudinal interior cavity, it is discernible that the supply helix 4 has a clear interior cross-section or a lumen such that a stylet 8, which is rotational in the operating position, can be inserted into the supply helix 4 and/or its interior longitudinal cavity and is inserted in the operating position according to FIGS. 3 and 4, and the stylet at its distal end 9, in a manner to be described, can be coupled to the rotatable holder 3 of the helical screw 2 or in a modified design to said helical screw 2 itself, and that this stylet 8 additionally can be coupled rotationally to the proximal end of the supply helix 4 or to the part engaging the supply helix 4 or the connector pin 6 in a manner to be described, which is particularly well discernible when comparing FIG. 1 to FIG. 3.

The proximal end of the stylet 8 is here provided with a sheath-like coupling, in its entirety marked 10, which according to FIGS. 1 and 3 fits onto the connector pin 6 connected to the supply helix 4 and which can be connected thereto via a clamping screw 11 in a detachable manner. In FIG. 1, this sheath-like coupling 10 is axially spaced apart from the connector pin 6 but already in a position in which it can be pushed onto the connector pin 6, with subsequently the detachably fastening occurs with the help of a clamping screw 11, as discernible in FIG. 3.

The sheath-like coupling 10 is therefore a clamping sheath 12 that can be detachably connected to the connector pin 6 via the clamping screw 11, which has an also sheath-shaped coupling appendage aligned in the operating position to the proximal end, which is oriented coaxially in reference to the clamping sheath 12 and also to the interior longitudinal cavity of the supply helix 4. The proximal end of the stylet 8 can be connected to this coupling appendage 13 in the direction of rotation in a power transmitting fashion, to be described in the exemplary embodiment.

In FIG. 1, in a side view, and in FIG. 3, partially in a longitudinal cross-section, a coupling sheath 14 is discernible at the proximal end of the stylet 8, which concentrically encompasses the end of the stylet 8, is connected thereto, and leaves a distance between its interior wall and the stylet 8, so that it can be attached in a force fitting and/or form fitting manner to the coupling appendage 13 of the sheath-like coupling 10. Thus, in the operating position this sheath-like coupling 10 fills the intermediate space between the stylet 8 and the interior wall of the coupling sheath 14 connected thereto, at least over a portion of its cross-section, and primarily with its exterior side effectively contacts and engages the interior side of said coupling appendage 13 (cf. FIG. 3).

According to FIGS. 2 and 4 the distal end 9 of the stylet 8 can be coupled in the rotational direction to the holder 3 for the helical screw 2 in a form-fitting manner, with this distal end 9 being flattened or embodied polygonal, for example square, hexagonal, or the like. At the holder 3, a matching coupling opening 15 is provided, into which the distal end 9 of the stylet 8 can be inserted in the axial direction. In FIG. 2, the end 9 has not yet been inserted into the coupling opening 15, while FIG. 4 shows its inserted and coupled position.

It remains to be stated that the appendage 13 of the sheath-like coupling engaging and clamping connector 12 engaging the connector pin 6 in the operating position in a detachable fashion and the interior cross-section of the coupling sheath 14 arranged at the proximal end of the stylet are profiled matching each other such that the coupling sheath 14 and the coupling appendage 13 can be telescopically moved or displaced in the axial direction, but are in a form-fitting engagement with each other in the rotational direction.

In order to improve the operation of the cardiac pacemaker—electrode 1, particularly to facilitate the rotational insertion of the helical screw 2 in the heart tissue, the coupling sheath 14 arranged at the proximal end of the stylet 8 is simultaneously embodied as a rotational handle and roughened or profiled at the outside. The user can therefore, after the insertion of the stylet 8 into the operating position shown in FIGS. 3 and 4, perform the rotational motion at this coupling sheath 14, by which simultaneously the holder 3 and thus the helical screw 2 and also the connector pin 6 can be made to rotate as desired together with the supply helix 4 so that the helical screw 2 can reach its operational position without the supply helix 4 alone providing the respective rotational motion and thus, based on its length and flexibility, was subject to substantial torque. Such an internal rotation or torque of the supply helix 4 is avoided in that a rotation of the stylet 8 occurs synchronous movement by the coupling sheath 14 serving as the rotational handle also being rotated, which leads to a rotation of the holder 3 and simultaneously also to the rotation of the supply helix 4. In this way the user can predetermine the insertion depth of the helical screw 2 into the tissue via the number of rotations at the coupling sheath 14 and also calculate it based on the incline of the helical screw 2, without being forced to control the insertion depth by any X-Ray examination.

The cardiac pacemaker—electrode 1 is provided at its distal end with a helical screw 2, which is connected to an electrically conductive supply screw 5, which in turn can be coupled via a connector 5 and a connector pin 6 to a cardiac pacemaker and which is rotational for rotating and axially adjusting the helical screw 2. A stylet 8 can be inserted through an interior longitudinal cavity of the supply helix 4, which at its distal end 9 can be directly or indirectly connected or coupled to the rotational helical screw 2, with the stylet 8 additionally being directly or indirectly coupled in a rotationally fixed or torque proof manner to the proximal end of the supply helix 4, so that when rotating the stylet 8 both the supply helix 4 as well as the helical screw 2 are rotated without any considerable torsion occurring of the supply helix 4.

1. A cardiac pacemaker—electrode (1) comprising a helical screw (2) that is rotationally displaceable from a retracted position into an active fastening position, and in the fastening position is engagable with heart tissue, the helical screw is connected to an electrically conductive supply helix (4), which in turn can be coupled to a cardiac pacemaker via a connector (5) and which in turn can be rotated for rotating and axially adjusting the helical screw (2), the supply helix (4) has a clear interior cross-section, a stylet (8) that is rotational in an operating position is inserted into the supply helix (4), the stylet has a distal end (9) that is couplable to a rotational holder (3) of the helical screw (2) in a form-fitting manner, and the stylet (8) additionally can be coupled in a rotationally fixed fashion to a proximal end of the supply helix (4) or to a part engaging the supply helix (4) or a connector pin (6) connected to the supply helix.

2. A cardiac pacemaker—electrode according to claim 1, wherein the proximal end of the stylet (8) is provided with a sheath-like coupling (10), which matches the connector pin (6) connected to the supply helix (4) and can be detachably connected thereto.

3. A cardiac pacemaker—electrode according to claim 2, wherein the sheath-like coupling (10) for the connector pin (6) is a clamping sheath (12) that is couplable thereto in a detachable fashion, and has a coupling appendage (13), that is aligned to a proximal end thereof in an operating position, by which a proximal end of the stylet (8) can be connected such that it transfers power in a rotational direction.

4. A cardiac pacemaker—electrode according to claim 3, wherein a coupling sheath (14) is provided at the proximal end of the stylet (8), which concentrically encompasses the end of the stylet (8) at a distance, and which can be attached to the coupling appendage (13) of the sheath-like coupling (10) in a force-fitting and/or form-fitting manner.

5. A cardiac pacemaker—electrode according to claim 4, wherein the distal end (9) of the stylet (8) can be coupled to the holder (3) for the helical screw (2) in a form-fitting manner in the rotational direction and is embodied flattened or polygonal, and a matching coupling opening (15) is provided at the holder (3) for the helical screw.

6. A cardiac pacemaker—electrode according to claim 5, wherein the coupling appendage (13) of the clamping sheath (12), detachably engaging the connector pin (6) in the operational position, and an interior cross-section of the coupling sheath (14) arranged at the proximal end of the stylet (8) are profiled matching each other so that the coupling sheath (14) and the coupling appendage (13) are telescopically displaceable in an axial direction and are form-fitting in the rotational direction.

7. A cardiac pacemaker—electrode according to one claim 6, characterized in that the coupling sheath (14) arranged at the proximal end of the stylet (8) simultaneously serves as a rotational handle and is roughened or profiled on an exterior thereof.
